SEQUENCE LISTING (3 sequences)

SEQ ID NO: 1 - protein, 4 aa, Artificial Sequence
  Synthetic Peptide
VLQS

SEQ ID NO: 2 - protein, 4 aa, Artificial Sequence
  Synthetic Peptide
VLNS

SEQ ID NO: 3 - protein, 5 aa, Artificial Sequence
  Synthetic Peptide
AAFAA

(19) United States
(12) Patent Application Publication
(10) Pub. No.: US 2023/0203558 A1
(43) Pub. Date: Jun. 29, 2023

(54) DIAGNOSTIC PEPTIDE FOR USE IN A METHOD OF DIAGNOSIS OF VIRAL INFECTION, KIT AND SYSTEM

(71) Applicant: Original G B.V., Groningen (NL)

(72) Inventors: Matthew Francis Burton, Groningen (NL); Joost Alexander Christiaan Gazendam, Groningen (NL)

(73) Assignee: Original G B.V., Groningen (NL)

(21) Appl. No.: 17/915,513

(22) PCT Filed: Mar. 30, 2021
(86) PCT No.: PCT/EP2021/058249

§ 371 Date: Sep. 29, 2022

(30) Foreign Application Priority Data
Mar. 30, 2020 (EP) 20166722.7
Aug. 19, 2020 (EP) 20191741.6
Nov. 17, 2020 (EP) 20208133.7

(51) Int. Cl. C12Q 1/04; C12Q 1/37; G01N 33/542

(52) U.S. Cl. CPC C12Q 1/04; C12Q 1/37; G01N 33/542; G01N 2333/165

(57) ABSTRACT

The present invention relates to a diagnostic peptide and methods, kits and systems for the in vitro diagnostic of an infection in a subject. The methods using the peptide of the invention comprise the steps of: i) providing a bodily fluid sample, ii) contacting the bodily fluid sample with a peptide comprising a fluorescent agent having an emission wavelength of 650-900 nm and a non-fluorescent agent having an absorption wavelength of 650-900 nm, for quenching said emission of said fluorescent agent, and a cleavage site located between said fluorescent agent and first non-fluorescent agent, the cleavage site being specific for a viral protease, iii) monitoring the fluorescence in the range of 650-900 nm from the peptide in step ii), wherein an increase in fluorescence in the range of 650-900 nm is indicative for the presence of a viral protease in the sample.

### REFERENCE TO AN ELECTRONIC SEQUENCE LISTING

The contents of the electronic sequence listing (sequencelisting.txt; Size: 671 bytes; and Date of Creation: Feb. 6, 2023) is herein incorporated by reference in its entirety.

### FIELD OF THE INVENTION

The present invention relates to a diagnostic peptide for use in a method of diagnosis in vitro for viral infection, a method for in vitro diagnosis of a viral infection in a subject, a kit for detecting the presence of an infection in a subject, and a system for detecting the presence of an infection in a subject, wherein the method comprises providing a sample of a bodily fluid.

### BACKGROUND ART

There is an increasing clinical need for improved diagnostic methods for viral infection in a patient. Rapid detection and identification of viral pathogens is essential to limit transfer and spread of viral disease and to monitor treatment methods. The need for such methods can be illustrated be the events surrounding the 2019/2020 global pandemic SARS-associated coronavirus.

SARS is a viral respiratory illness caused by the SARS-associated coronavirus (SARS-CoV). SARS was first reported in Asia in February 2003. In 2019/20 a pandemic of SARS-CoV-19 caused >600,000 deaths worldwide.

The SARS coronavirus belongs to a group of viruses similar to those causing the common cold. The SARS virus spreads by close person-to-person contact and is thought to be transmitted most readily by respiratory droplets produced when an infected person coughs or sneezes. Droplet spread can occur when droplets from an infected person are propelled a short distance (generally up to 3 feet) through the air and deposited on the mucus membranes of nearby persons. The virus also can spread when a person touches a surface contaminated with infectious droplets and then touches his or her mouth, nose, or eyes.

Real-time reverse transcriptase-PCR (RT-PCR) detection is currently favoured for the detection of coronavirus because of its advantages as a specific, and simple quantitative assay. Moreover, real-time RT-PCR is more sensitive than the conventional RT-PCR assay, which help much for the diagnosis in early infection. Therefore, the real-time RT-PCR assay still is a predominant method to be applied for the detection of all kinds of coronaviruses including SARS-CoV-2 [14].

Even so, there is a need to improve the real-time RT-PCR assay. Since the RT-PCR methods are prone to contamination and require time-consuming sample handling and post-PCR analysis, an improved assay is a TaqMan-based real-time RT-PCR that can easily be implemented in the routine diagnostic setting for the detection of HCoV. Moreover, to further improve the sensitivity, a real-time quantitative RT-PCR assay for SARS-CoV can be carried our with the use of 2 TaqMan probes, instead of 1 probe. This simple modification using dual TaqMan probes for quantification has wide applications in areas in which ultrasensitivity is critically required, with the SARS-CoV detection limit of 1 copy RNA per reaction.

LAMP is a novel isothermal nucleic acid amplification method with high efficiency. However, analysis of the results is typically by time consuming gel electrophoresis. If the methods rely on nonspecific signal transduction schemes, such as the fluorescence dyes intercalation into any double-stranded DNA amplicons, or solution turbidity due to the release of pyrophosphates during polymerization, the possibility of unexpected signals derived from primer dimer or non-primer reactions cannot be exclude,

In clinical detection, the lack of safe and stable external positive controls (EPC) could become a serious problem in the diagnosis of coronavirus by PCR/LAMP methods.

Meanwhile, the rapidly mutating nature of coronaviruses highlights the need for accurate detection of genetically diverse coronaviruses.

There is a need for a rapid, diagnostic method that identifies the presence of a viral infection in a bodily fluid sample and that can be carried out at the point of care.

### SUMMARY OF THE INVENTION

The present invention provides a diagnostic peptide for use in a method of diagnosis in vitro for viral infection, the peptide represented by formula (I)

[a]-[b]-[c] (I)

wherein:

[a] is a fluorescent agent having an emission wavelength of 650-900 nm,

[b] is a peptide comprising the amino acid sequence Xₐₐ₁, Xₐₐ₂, Xₐₐ₃

wherein Xₐₐ₁ is a hydrophobic or basic amino acid,
wherein Xₐₐ₂ is a polar, neutral or basic amino acid,
wherein Xₐₐ₃ is a polar, neutral or basic amino acid,

[c] is a non-fluorescent agent having an absorption wavelength of 650-900 nm, for quenching said emission of said fluorescent agent,

wherein Xₐₐ₁, Xₐₐ₂, Xₐₐ₃ represents a cleavage site for a viral encoded protease, wherein cleavage of the cleavage site results in release of the non-fluorescent agent from the peptide, wherein release of the non-fluorescent agent is indicative for the presence of a virus, preferably wherein [b] represents at most 30% by weight of the total mass of the diagnostic peptide, based on the total mass of [a]-[b]-[c].

In some jurisdictions, the present invention can be defined as a method for in vitro diagnosing a viral infection using a diagnostic peptide represented by formula (I)

[a]-[b]-[c] (I)
wherein:
[a] is a fluorescent agent having an emission wavelength of 650-900 nm,
[b] is a peptide comprising the amino acid sequence Xₐₐ₁, Xₐₐ₂, Xₐₐ₃
wherein Xₐₐ₁ is a hydrophobic or basic amino acid,
wherein Xₐₐ₂ is a polar, neutral or basic amino acid,
wherein Xₐₐ₃ is a polar, neutral or basic amino acid,
[c] is a non-fluorescent agent having an absorption wavelength of 650-900 nm, for quenching said emission of said fluorescent agent,
wherein Xₐₐ₁, Xₐₐ₂, Xₐₐ₃ represents a cleavage site for a viral encoded protease, wherein cleavage of the cleavage site results in release of the non-fluorescent agent from the peptide, wherein release of the non-fluorescent agent is indicative for the presence of a virus, preferably wherein [b] represents at most 50% by weight of the total mass of the diagnostic peptide, based on the total mass of [a]-[b]-[c].

Likewise, in some jurisdictions, the present invention can be defined as the use of a diagnostic peptide in the in vitro diagnosis of viral infection, wherein the peptide is represented by formula (I)

[a]-[b]-[c] (I)
wherein:
[a] is a fluorescent agent having an emission wavelength of 650-900 nm,
[b] is a peptide comprising the amino acid sequence Xₐₐ₁, Xₐₐ₂, Xₐₐ₃
wherein Xₐₐ₁ is a hydrophobic or basic amino acid,
wherein Xₐₐ₂ is a polar, neutral or basic amino acid,
wherein Xₐₐ₃ is a polar, neutral or basic amino acid,
[c] is a non-fluorescent agent having an absorption wavelength of 650-900 nm, for quenching said emission of said fluorescent agent,
wherein Xₐₐ₁, Xₐₐ₂, Xₐₐ₃ represents a cleavage site for a viral encoded protease, wherein cleavage of the cleavage site results in release of the non-fluorescent agent from the peptide, wherein release of the non-fluorescent agent is indicative for the presence of a virus, preferably wherein [b] represents at most 50% by weight of the total mass of the diagnostic peptide, based on the total mass of [a]-[b]-[c].

The invention further refers to an in vitro method for the diagnosis of viral infection in a subject, the method comprising the steps of:

i) contacting a bodily fluid/tissue sample with a peptide comprising a fluorescent agent having an emission wavelength of 650-900 nm and a non-fluorescent agent having an absorption wavelength of 650-900 nm, for quenching said emission of said fluorescent agent, and a cleavage site located between said fluorescent agent and first non-fluorescent agent, the cleavage site being specific for a viral protease,

ii) monitoring the fluorescence in the range of 650-900 nm from the peptide in step ii),

wherein an increase in fluorescence in the range of 650-900 nm is indicative for the presence of a viral protease in the sample.

According to the present invention, a method which is defined herein enables rapid and specific detection of infection in a subject by analysing bodily fluid/tissue samples. In the presence of a viral protease that recognises and cleaves the cleavage site, the first non-fluorescent agent is released from the reagent and as a result a fluorescent signal is emitted, which can be detected by a suitable detector. It has been surprisingly found that the detection of viral protease by monitoring wavelengths between 650-900 nm provides an improved limit of detection compared to prior art methods. The method is simple to carry out and amenable to point of care use.

An advantage of the present method is that it can be carried out in a bodily fluid sample without the need to enrich the cell count by, for example, an enrichment step. As a result, the present method is able to detect viral infection rapidly, for example, within minutes of contacting the reagent with sample.

The method overcomes the problems of the lack of safe and stable external positive controls (EPC) in PCR/LAMP methods.

In a further aspect there is provided a kit for the diagnostic of a viral infection in a subject, comprising:

a) a container comprising the diagnostic peptide of formula (I),

b) a set of instructions for carrying out the diagnostic method defined herein.

In yet a further aspect, there is provided a system for the diagnosis of a viral infection in a subject, comprising:

a) a container for receiving a sample,

b) a container comprising the diagnostic peptide of formula (I),

c) a device adapted to receive the container and monitor the fluorescence signal emitted from the peptide when the peptide is contacted with the sample of bodily fluid.

The present invention also refers to a diagnostic peptide represented by formula (Ib)

[a]-[linker₁]-[b]-[-linker₂]-[c] (Ib)

wherein:

[a] is a fluorescent agent having an emission wavelength of 650-900 nm,

[b] is a peptide comprising 3-10 amino acids and having the amino acid sequence Xₐₐ₁, Xₐₐ₂, Xₐₐ₃

wherein Xₐₐ₁ is histidine, arginine or lysine,

wherein Xₐₐ₂ is glutamine,

wherein Xₐₐ₃ is a polar, neutral or basic amino acid,

[c] is a non-fluorescent agent having an absorption wavelength of 650-900 nm, for quenching said emission of said fluorescent agent,

[linker₁] and [linker₂] are independently selected from the group of an optionally substituted hydrocarbyl group and a non-proteolytic hydrocarbyl group

wherein Xₐₐ₁, Xₐₐ₂, Xₐₐ₃ represents a cleavage site for a viral encoded protease,

wherein cleavage of the cleavage site results in release of the non-fluorescent agent from the peptide, wherein release of the non-fluorescent agent is indicative for the presence of a virus belonging to the family of Coronaviridae, preferably wherein the virus is SARS-Cov-2 (SARS-COV-19).

The present invention further relates to methods, uses, kits and systems using the diagnostic peptide as defined herein.

### DESCRIPTION OF EMBODIMENTS

The term “subject” as used herein means an animal or human individual who is at risk of or suspected of having an infection. The terms “patient,” “subject,” “individual,” and the like are used interchangeably herein, and refer to any animal or human amenable to the methods described herein. In certain non-limiting embodiments, the patient, subject or individual is a human.

The term “bodily fluid sample” as used herein means biological material isolated from a subject. The step of obtaining the sample is not part of the present invention.

The term “viral protease” as used herein means a peptidase that is encoded by viral RNA.

The terms “protease” and “peptidase” as used herein mean viral RNA encoded polypeptides that are capable of cleaving a peptide bond (C(O)NH) in a protein at a cleavage site (amino acid motif).

The term “peptide” as used herein means an oligomer comprising at least 3 amino acids. Preferably, the peptide comprises no more than 20 amino acids. Preferably the peptide comprises between 4 and 20 amino acids, more preferably between 6 and 15 amino acids. In one embodiment, the peptide comprises preferably 3 to 10 amino acids.

The amino acids used may be any amino acid, preferably chosen from the group of naturally occurring amino acids or from the group of synthetic amino acids, in particular derivatives of natural amino acids. Preferably, the peptide includes a “non-natural amino acid motif” which comprises the cleavage site for viral proteases. “Non-natural amino acid motif” as used herein refers to non-naturally occurring sequences, i.e., genetically engineered sequences and sequences derived from molecular modelling.

The term “monitoring”, “measuring” “measurement,” “detecting” or “detection” or “diagnostic”, as used herein means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The term “viral encoded protease” means a protease that is translated from the genetic information (RNA) of a viral genome.

The term “infection” as used herein means a clinically relevant viral load. An infection may be symptomatic or asymptomatic in a subject. A clinically relevant viral load may result in a symptomatic or asymptomatic individual.

In a first aspect, there is provided a diagnostic peptide for use in a method of diagnosis in vitro for viral infection the peptide represented by formula (I)

[a]-[b]-[c] (I)

wherein:

[a] is a fluorescent agent having an emission wavelength of 650-900 nm,

[b] is a peptide comprising the amino acid sequence Xₐₐ₁, Xₐₐ₂, Xₐₐ₃

wherein Xₐₐ₁ is a hydrophobic or basic amino acid,
wherein Xₐₐ₂ is a polar, neutral or basic amino acid,
wherein Xₐₐ₃ is a polar, neutral or basic amino acid,

[c] is a non-fluorescent agent having an absorption wavelength of 650-900 nm, for quenching said emission of said fluorescent agent,

wherein Xₐₐ₁, Xₐₐ₂, Xₐₐ₃ represents a cleavage site for a viral encoded protease, wherein cleavage of the cleavage site results in release of the non-fluorescent agent from the peptide, wherein release of the non-fluorescent agent is indicative for the presence of a virus, preferably wherein [b] represents at most 50% by weight of the total mass of the diagnostic peptide, based on the total mass of [a]-[b]-[c].

The term “Hydrophobic amino acid” means an amino acid that primarily has a side chain that confers a tendency to associate in an aqueous environment. The terms “hydrophobic” and “hydrophobicity” are well understood by the skilled person, as known from the handbook ‘IUPAC Gold Book’ IUPAC. Compendium of Chemical Terminology, 2nd ed. (the “Gold Book”). Compiled by A. D. McNaught and A. Wilkinson. Blackwell Scientific Publications, Oxford (1997). https://doi.org/10.1351/goldbook.HT06964. Examples of hydrophobic amino acids are alanine, leucine, valine, norleucine, norvaline, isoleucine, isovaline, alloisoleucine, phenylalanine, proline, methionine, and tryptophan.

The term “polar, neutral amino acid” means an amino acid capable of forming one or more hydrogen bonds”. Examples of polar, neutral amino acids are serine, threonine, cysteine, asparagine, glutamine, and tyrosine.

The term “basic amino acid” means an amino acid that have basic side chains at neutral pH. Examples of basic amino acids are arginine, lysine and histidine.

Xaa₁ is a hydrophobic or basic amino acid. Preferably Xaa₁ is selected form the group consisting of Xaa₁ is selected from the group consisting of alanine, leucine, valine, norleucine, norvaline, isoleucine, isovaline, alloisoleucine, phenylalanine, histidine, arginine and lysine, more preferably from the group consisting of histidine, lysine and arginine. Preferably, Xaa₁ is histidine.Xaa₂ is a polar, neutral or basic amino acid. Preferably, Xaa₂ is selected from the group consisting of histidine, asparagine and glutamine, more preferably Xaa₂ is selected from the group consisting of histidine and glutamine. Preferably, Xaa₂ is glutamine.

Xaa₃ is a polar, neutral or basic amino acid. Preferably, Xaa₃ is selected from the group consisting of serine, threonine and glycine. More preferably Xaa₃ is selected from the group consisting of serine and threonine, even more preferably Xaa₃ is serine.

Preferably, the amino acid sequence Xaa₁,Xaa₂,Xaa₃ has a sequence selected from LQS, HQS, FHT, NleQS, NvaQS, VLQS, (SEQ ID NO:1), VLNS (SEQ ID NO:2), RQS or KQS.

The peptide preferably comprises between 4 and 10 amino acids, more preferably between 5 and 9 amino acids.

Preferably, the cleavage site consists of between 3 and 8 amino acids, preferably between 4 and 7 amino acids. cleavage site preferably comprises of a number of amino acids, for example at least two, preferably at least three amino acids, more preferably at least four amino acids, even more preferably at least five amino acids.

Preferably [b] represents at most 50% by weight of the total mass of the diagnostic peptide, based on the total mass of [a]-[b]-[c]. More preferably, [b] represents at most 45% by weight of the total mass of the diagnostic peptide, even more preferably at most 40% by weight of the total mass of the diagnostic peptide, yet more preferably at most 35% by weight of the total mass of the diagnostic peptide, even yet more preferably at most 30% by weight of the total mass of the diagnostic peptide, most preferably at most 25% by weight of the total mass of the diagnostic peptide, based on the total mass of [a]-[b]-[c].

Preferably, [b] represents between 5 and 50% by weight of the total mass of the diagnostic peptide, more preferably between 7 and 45% by weight, even more preferably between 10 and 40% by weight, even more preferably between 15 and 35% by weight, of the total mass of the diagnostic peptide, based on the total mass of [a]-[b]-[c].

Preferably, the increase in fluorescence is determined as a relative increase, whereby the fluorescence measured in the presence of the diagnostic peptide and a sample (F1) is subtracted from the fluorescence of the diagnostic peptide in the absence of a sample (F2). Preferably, the relative increase is represented as a proportion of the fluorescence of the diagnostic peptide in the absence of a sample (F2), according to the equation:

percent relative fluorescence=[(F1−F2)/F2]*100

Preferably the amino acid sequence Xaa1,Xaa2,Xaa3 is flanked by further amino acids, hydrocarbyl moieties and/or non-proteolytic hydrocarbyl linkers.

In a preferred embodiment, the hydrocarbyl linker is selected from the group consisting of beta-alaninyl, 4-aminobutyrl, 2-(aminoethoxy)acetyl, 3-(2-aminoethoxy)propanyl, 5-aminovaleryl, 6-aminohexyl, 8-amino-3,6-dioxaoctanyl and 12-amino-4,7,10-trioxadodecanyl, preferably 6-aminohexyl.

According to one embodiment, the diagnostic peptide is represented by the formula (Ia):

[a]-[linker₁]-[b]-[-linker₂]-[c] (Ia)

wherein the linkers are independently selected from the group consisting of an optionally substituted hydrocarbyl group, and a non-proteolytic hydrocarbyl group.

In one aspect of the invention, the diagnostic peptide is represented by formula (Ib)

[a]-[linker1]-[b]-[-linker2]-[c] (Ib)
wherein:
[a] is a fluorescent agent having an emission wavelength of 650-900 nm,
[b] is a peptide comprising 3-10 amino acids and having the amino acid sequence Xaa1,Xaa2,Xaa3
wherein Xaa1 is histidine, arginine or lysine,
wherein Xaa2 is glutamine,
wherein Xaa3 is a polar, neutral or basic amino acid,
[c] is a non-fluorescent agent having an absorption wavelength of 650-900 nm, for quenching said emission of said fluorescent agent,

[linker1] and [linker2] are independently selected from the group of an optionally substituted hydrocarbyl group and a non-proteolytic hydrocarbyl group

wherein Xaa1,Xaa2,Xaa3 represents a cleavage site for a viral encoded protease, wherein cleavage of the cleavage site results in release of the non-fluorescent agent from the peptide, wherein release of the non-fluorescent agent is indicative for the presence of a virus preferably belonging to the family of Coronaviridae. More preferably, the virus is SARS-Cov-2 (SARS-COV-19).

The peptide of formula (Ib) can also be defined by its use in the in vitro method of diagnosis of an infection by SARS-Cov-2 (SARS-COV-19).

The fluorescent agent having an emission wavelength of 650-900 nm is preferably a cyanine moiety (dye). Preferably, the non-fluorescent agent having an emission wavelength of 650-900 nm is a cyanine moiety (dye).

Preferably, the fluorescent agent is a cyanine dye having an emission wavelength of 650-900 nm and the non-fluorescent agent is a cyanine dye having an absorption wavelength of 650-900 nm.

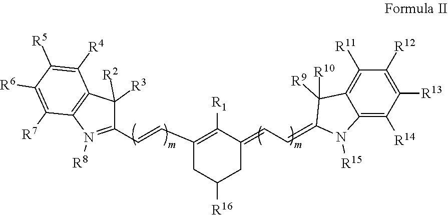

In an embodiment, the first fluorescent agent is a cyanine dye having the general formula as shown in formula II, wherein R¹ is selected from the group consisting of H, halo, and

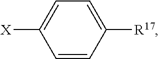

where R¹⁷ is selected from the group consisting of carboxyl, amino and sulfanato; X is selected from the group consisting of O, S, NH and N-hydrocarbyl; R², R³, R⁹, R¹⁰ are each independently selected from the group consisting of H and hydrocarbyl; R⁴, R⁵, R¹¹, R¹² are each independently selected from the group consisting of H, hydrocarbyl and sulfanato or together with the atoms to which they are bonded form an aromatic ring; R⁶, R⁷, R¹³, R¹⁴ are each independently selected from the group consisting of H and hydrocarbyl, R⁸ and R¹⁵ are each independently selected from the group consisting of hydrocarbyl, (CH₂)qFG or (CH₂)_{P}LN wherein at least one of R⁸ and R¹⁵ is (CH₂)qFG, wherein q is an integer from 1 to 20 and FG is a functional group that does not directly react with carboxyl, hydroxyl, amino or thiol groups, wherein p is an integer from 1 to 20 and LN is a linker group that reacts with carboxyl, hydroxyl, amino or thiol groups; R¹⁶ is H or hydrocarbyl.

Preferably, the fluorescent agent is an agent wherein R¹ is

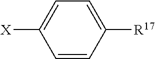

wherein X is O and R¹⁷ is SO₃Na; R², R³, R⁹, R¹⁰ are hydrocarbyl, preferably methyl; R⁴ and R¹¹ are H and R⁵ and R¹² are H or sulfanato; R⁶, R⁷, R¹³, R¹⁴ are H; R8 is (CH₂)qFG where q is 4 and FG is sulfanato; R¹⁵ is (CH₂)_{P}LN where p is 5 and LN is carboxyl, R¹⁶ is H.

Even more preferably, the fluorescent agent is an agent wherein R¹ is

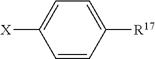

wherein X is O and R¹⁷ is SO₃Na; R², R³, R⁹, R¹⁰ are methyl; R⁴ and R¹¹ are H and R⁵ and R¹² are sulfanato; R⁶, R⁷, R¹³, R¹⁴ are H; R8 is (CH₂)qFG where q is 4 and FG is sulfanato; R¹⁵ is (CH₂)_{P}LN where p is 5 and LN is carboxyl, R¹⁶ is H. Preferably, the fluorescent agent is an agent corresponding to formula III.

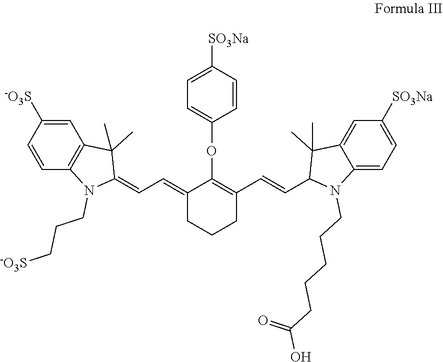

The non-fluorescent agent having an absorption wavelength of 650-900 nm, is a compound that has little or no intrinsic fluorescence and which can efficiently quench the fluorescence from a proximate fluorophore with little background. In an embodiment the non-fluorescent agent is a cyanine molecule. Cyanine molecules, also referred to as cyanine dyes, include compounds having two substituted or unsubstituted nitrogen-containing heterocyclic rings joined by a polymethine chain.

In a preferred embodiment, the non-fluorescent agent is an agent wherein R¹ is chloro, R², R³, R⁹, R¹⁰ are methyl; R⁴ is H and R⁵ is N-hydrocarbyl, preferably N[(CH₂)₃SO₃Na]₂; R¹¹ and R¹² form an aromatic ring monosubstituted with sulfanato group; R⁶, R⁷, R¹³, R¹⁴ are H; R³ is (CH₂)qFG where q is 3 and FG is sulfanato; R¹⁵ is (CH₂)_{P}LN where p is 5 and LN is carboxyl; R¹⁶ is H.

In another embodiment, the fluorescent agent and the non-fluorescent are the same agent, preferably wherein R¹ is

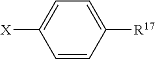

in X is O and R¹⁷ is SO₃Na, R², R³, R⁹, R¹⁰ are hydrocarbyl, preferably methyl, R⁴, R⁵, R¹¹, R¹² are H, R⁶, R⁷, R¹³, R¹⁴ are H, R⁸ is (CH₂)qFG where q is 4 and FG is sulfanato, R¹⁵ is (CH₂)PLN where p is 5 and LN is carboxyl, R¹⁶ is H.

The non-fluorescent agent may also be a quenching moiety for example BHQ3, (Biosearch) QC-1 (Li-COR.com), TF7QWS, TF8QWS (AAT Bio) or particles comprising such compounds, for example gold nanoparticles and ferro-nanoparticles. In an embodiment, the peptide substrate is a nanoparticle comprising a peptide as defined herein. In a preferred embodiment, the non-fluorescent agent is QC-1 (Li-COR.com).

Examples of fluorescent agents that can be used with in present invention include, but are not limited to, Alexa Fluor® 660, Alexa Fluor® 680, Alexa Fluor® 700, Alexa Fluor® 750 ATTO 680, ATTO 700, DY-647, DY-650, DY-673, DY-675, DY-676, DY-680, DY-681, DY-682, DY-690, DY-700, DY-701, DY-730, DY-731, DY-732, DY-734, DY-750, DY-751, DY-752, DY-776, DY-781, DY-782, DY-831, La Jolla Blue, Cy5, Cy5.5, Cy7, IRDye® 8000W, IRDye® 38, IRDye® 800RS, IRDye® 700DX, IRDye® 680, and TF7WS (AATBio) among others. “Alexa Fluor” dyes are available from Molecular Peptides Inc., Eugene, Oreg., U.S.A. (www.peptides.com). “ATTO” dyes are available from ATTO-tec GmbH, Siegen, Germany (www.atto-tec.com). “DY” dyes are available from Dyomics GmbH, Jena, Germany (www.dyomics.com). La Jolla Blue is available from Hyperion Inc. “Cy” dyes are available from Amersham Biosciences, Piscataway, N.J., U.S.A. (www.amersham.com).” IRDye® infrared dyes” are available from LI-COR® Bioscience, Inc. Lincoln, N E, U.S. A (www.licor.com).

In a preferred embodiment, the fluorescent agent is IRdye8000W, Cy7 or TF7WS, preferably Cy7. Preferably, the Cy7 has the structure according to Formula IV:

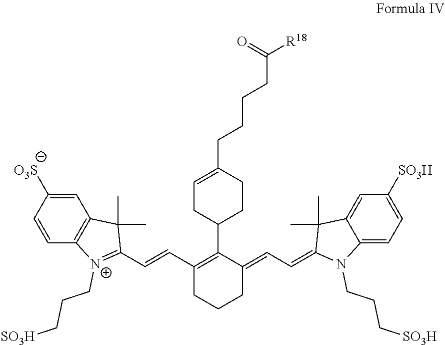

Where R¹⁸ is a linker group that reacts with carboxyl, hydroxyl, amino or thiol groups. Cy7 is for example available from ClickChemistryTools; [https:/clickchemistrytools.com/product/cy7-nhs-ester/].

Preferably, the peptide has the structure ([a]-[b]-[c])^{m,p} where m is an integer between 1 and 8 and p is either +(plus) or −(minus). Preferably, the peptide has the structure ([a]-[b]-[c])^{m,p} where m in an integer between 1 and 8 and p is minus, more preferably, m in an integer between 2 and 7 and p is minus, even more preferably m in an integer between 3 and 6 and p is minus, most preferably m in an integer between 3 and 6 and p is minus.

Preferably, the peptide has the structure ([a]-[b]-[c])^{m,p} where m in an integer between 1 and 8 and p is plus, more preferably, m in an integer between 2 and 7 and p is plus, even more preferably m in an integer between 3 and 6 and p is plus, most preferably m in an integer between 3 and 6 and p is plus.

Preferably, the cleavage site consists of between 3 and 8 amino acids, preferably between 4 and 7 amino acids. The cleavage site preferably comprises of a number of amino acids, for example at least two, preferably at least three amino acids, more preferably at least four amino acids, even more preferably at least five amino acids. Preferably, the cleavage site has the structure XYZ, wherein X is at least one amino acid, Y is a portion of molecular structure composed of at least two amino acids, and Z is at least one amino acid. X and Z may be any amino acid. Preferably Y comprises a dipeptide consisting of an aliphatic, hydrophobic amino acid and an aromatic or cyclic amino acid or a basic amino acid and a hydrophilic amino acid. The aliphatic hydrophobic amino acid is preferably selected from the group consisting of glycine, alanine, leucine, valine and derivatives thereof. The aromatic or cyclic amino acid is preferably selected from the group consisting of phenylalanine, tyrosine, tryptophan, proline and derivatives thereof. The basic amino acid is preferably lysine and the hydrophilic amino acid is preferably threonine or serine. Preferably, the cleavage site comprises non-natural amino acids. Non-natural amino acids are known to the skilled person.

Preferably, the peptide comprises a cell penetrating moiety. The cell-penetrating moiety comprises an amino acid sequence consisting of X1,X2X3X4X5X6X7X8,X9, wherein X1 is A, L or G or R, X2 is W, A, L or G or R, X3 is R or K, X4 is R, K, L or S, X5 is R, K, L, X6 is R, K, L, X7 is R, K, L, X8 is A, V, R, K, L, S or Q, and X9 is A, V, R, K, L, S Q, W, F or Y. Preferably, the cell penetrating moiety is an nona-arginine peptide where X is R for all X1,X2X3X4X5X6X7X8,X9.

As used herein, a “cell-penetrating peptide”, is a molecule, the core of which is a peptide. Other chemical groups can however be covalently bound to said peptidic core, in order to improve the overall stability of the molecule, and/or to provide it with additional properties, such as targeting ability. For example, a cell-penetrating peptide according to the invention can further comprise, covalently linked to the C-terminus, one or several groups chosen amongst a cysteamide, a cysteine, a thiol, an amide, a carboxyl, a linear or ramified C1-C6 alkyl optionally substituted, a primary or secondary amine, an osidic derivative, a lipid, a phospholipid, a fatty acid, a cholesterol, a poly-ethylene glycol, a nuclear localization signal (NLS), and/or a targeting molecule. Alternatively or additionally, the cell-penetrating peptide also comprises, covalently linked to the N-terminal end, one or several chemical entities chosen amongst an acetyl, a fatty acid, a cholesterol, a poly-ethylene glycol, a nuclear localization signal, and/or a targeting molecule. If necessary, for example in the case of N-terminal addition of cholesterol, a peptidic bridge can be used to bind a non-peptidic molecule.

Preferably, [a] is a cyanine moiety having an emission wavelength of 700-850 nm, [b] is a peptide comprising a sequence selected from LQS, HQS, RQS, KQS, FHT, NleQS, NvaQS, VLQS, (SEQ ID NO:1), VLNS (SEQ ID NO:2), more preferably a sequence selected from HQS, RQS or KQS, [linker1] and [linker2] are both 3-(2-aminoethoxy)propanyl, the [c] is a cyanine moiety having an absorption wavelength of 700-850 nm is a cyanine moiety.

Preferably the subject is a human or animal. Preferably, the subject is human.

Preferably, the subject is an animal. Preferably the subject is an animal and belongs to a species selected from the group consisting of Sus scrofa domesticus, Gallus Gallus domesticus, Felis catus, Canis lupus familiaris, Bos taurus, Ovis aries, Capra and aegagurus hircus, preferably a species selected from the group consisting of Sus scrofa domesticus, Gallus Gallus domesticus, Felis catus, Canis lupus familiaris, Bos taurus, more preferably a species selected from the group consisting of Sus scrofa domesticus, Gallus Gallus domesticus, Felis catus, even more preferably a species selected from the group consisting of Sus scrofa domesticus and Gallus Gallus domesticus, most preferably of the species Sus scrofa domesticus.

Preferably, the viral infection is caused by a virus belonging to the family of Picornaviridae, Coronaviridae, Adenoviridae, Coronaviridae. Preferably the virus is a Picornaviridae selected from the group consisting of Rhinovirus, a Hepatitis A, B, C, D or E virus.

Preferably, the rhinovirus is a virus of the genus Enterovirus.

Preferably the virus is a Adenoviridae virus selected from Atadenovirus, Aviadenovirus, Ichtadenovirus, Mastadenovirus, Siadenovirus and combinations thereof.

Preferably, the virus is a Coronaviridae selected from the genera Alphacoronavirus, Betacoronavirus, Gammacoronavirus, Deltacoronavirus. Preferably, the Coronavidae is a Alphacoronavirus or Betacoronavirus.

Preferably, the virus belongs to the genus Alphavoronavirus and is a species selected from the group consisting of Alphacoronavirus 1 (TGEV, Feline coronavirus, Canine coronavirus), Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2 and Scotophilus bat coronavirus 512. Preferably, the virus is Porcine epidemic diarrhea virus. Preferably, the virus is Porcine epidemic diarrhea virus and the subject belongs to Sus scrofa domesticus.

Preferably, the virus belongs to the genus Betacoronavirus and is a species selected from the group consisting of Betacoronavirus 1 (Bovine Coronavirus, Human coronavirus OC43), Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus (SARS-CoV, SARS-CoV-2) and Tylonycteris bat coronavirus HKU4. Preferably, the virus is Severe acute respiratory syndrome-related coronavirus (SARS-CoV, SARS-CoV-2), more preferably the virus is Severe acute respiratory syndrome-related coronavirus (SARS-CoV-2). Preferably, the virus is Severe acute respiratory syndrome-related coronavirus (SARS CoV, SARS-CoV-2) and the subject is human.

Preferably, the virus belongs to the genus Gammacoronavirus and is a species selected from the group consisting of Avian coronavirus and Beluga whale coronavirus SW1. Preferably, the virus is Avian coronavirus. Preferably, the virus is Avian coronavirus and the subject belongs to Gallus gallus domesticus.

Preferably, the virus belongs to the genus Deltacoronavirus and is a species selected from the group consisting of Bulbul coronavirus HKU11 and Porcine coronavirus HKU15. Preferably, the virus is Porcine coronavirus HKU15. Preferably, the virus is Porcine coronavirus HKU15 and the subject belongs to Sus scrofa domesticus

Preferably, the viral protease is encoded by viral RNA. The viral protease is preferably selected from the group consisting of Hepatitis encoded serine protease or metallo-protease (NS2 and NS3), Rhinovirus genome encoded cysteine proteases (3C and 2A), a 3C protease, Coronavirus genome encoded cysteine protease (3CL), an Adenoviruses genome encoded serine-centered, neutral protease, a Retroviruses encoded aspartyl protease. Preferably the viral protease is a 3C like (3CL) protease. Preferably wherein the Coronavirus protease is a hSARS Coronavirus genome encoded cysteine protease, SARS coronavirus 19 main protease (EC number 3.4.22.69, BRENDA database Release 2021.1 (January 2021)).

Preferably the protease is a SARS genome encoded cysteine protease, wherein the SARS protease is SARS coronavirus 19 main protease (EC number 3.4.22.69, BRENDA database Release 2021.1 (January 2021)). The main protease operates at no less than 11 cleavage sites on the large polyprotein lab (replicase lab, −790 kDa); the cleavage site is Leu-Gln=Ser-Ala-GI (where=is the scissile peptide bond).

Preferably, the viral encoded protease is a protease having at least 50% homology, preferably at least 55%, more preferably at least 60%, even more preferably 70%, yet more preferably at least 75%, even yet more preferably 80%, even yet more than 85%, even yet more preferably at least 90%, even yet more preferably at least 95%, preferably at least 97%, most preferably at least 98% to the amino acid sequence of 3C-like proteinase [Severe acute respiratory syndrome coronavirus 2] having accession number YP_009725301, version numberYP_009725301.1, 18 Jul. 2020 NCBI Protein Database GenPept [2021 Mar. 26 ]; available from https://www.ncbi.nlm.nih.gov/protein/1802476809.

Preferably, the viral encoded protease is a protease having at least 50% homology, preferably at least 55%, more preferably at least 60%, even more preferably 70%, yet more preferably at least 75%, even yet more preferably 80%, even yet more than 85%, even yet more preferably at least 90%, even yet more preferably at least 95%, preferably at least 97%, most preferably at least 98% to the amino acid sequence of 3C like protease (3 CL pro; R1A_SARS) at positions 3241-3546 having accession number P0C6U8 (Uniprot) Integrated into UniProtKB/Swiss-Prot: Sequence update: Jun. 10, 2008, Last modified: Feb. 10, 2021, Version 104 [2021 Mar. 26]; available from https://www.uniprot.org/uniprot/P0C6U8

Preferably, the viral encoded protease is a protease having at least 50% homology, preferably at least 55%, more preferably at least 60%, even more preferably 70%, yet more preferably at least 75%, even yet more preferably 80%, even yet more than 85%, even yet more preferably at least 90%, even yet more preferably at least 95%, preferably at least 97%, most preferably at least 98% to the amino acid sequence of 3C proteinase [Human rhinovirus A1] having accession number YP_009508983.1, version number YP_009508983.1, 24 Aug. 2018 NCBI Protein Database GenPept [2021 Mar. 26]; https://www.ncbi.nlm.nih.gov/protein/YP_009508983.

Preferably, the viral encoded protease is a protease having at least 50% homology, preferably at least 55%, more preferably at least 60%, even more preferably 70%, yet more preferably at least 75%, even yet more preferably 80%, even yet more than 85%, even yet more preferably at least 90%, even yet more preferably at least 95%, preferably at least 97%, most preferably at least 98% to the amino acid sequence of PEDV main proteinase [Porcine epidemic diarrhea virus] having accession number 4XFQ_A, PDB database, Deposited 28 Dec. 2014, Version 1.0 2016 Jan. 20 [2021 Mar. 26]; https://www.rcsb.org/structure/4XFQ.

Homology typically is measured using sequence analysis software, for example, the Sequence Analysis software package of the Genetics Computer Group, University of Wisconsin Biotechnology Center, 1710 University Avenue, Madison, Wis. 53705, MEGAlign (DNAStar, Inc., 1228 S. Park St., Madison, Wis. 53715), and MacVector (Oxford Molecular Group, 2105 S. Bascom Avenue, Suite 200, Campbell, Calif. 95008), BLAST as available via National Center for Biotechnology Information (NCBI)[Internet]. Bethesda (Md.): National Library of Medicine (US), National Center for Biotechnology Information; [2021]—https://www.ncbi.nlm.nih.gov/(Madden, T. L., Tatusov, R. L. & Zhang, J. (1996) “Applications of network BLAST server” Meth. Enzymol. 266:131-141). Such software matches similar sequences by assigning degrees of homology to various substitutions, deletions, and other modifications. Conservative substitutions typically include substitutions within the following groups: glycine and alanine; valine, isoleucine and leucine; aspartic acid, glutamic acid, asparagine, and glutamine; serine and threonine; lysine and arginine; and phenylalanine and tyrosine. Substitutions may also be made on the basis of conserved hydrophobicity or hydrophilicity (Kyte and Doolittle, J. Mol. BioL 157: 105-132, 1982), or on the basis of the ability to assume similar polypeptide secondary structure (Chou and Fasman, Adv. Enzymol. 47:45-148, 1978).

Preferably, the viral encoded protease is a protease having at least 50% sequence similarity, preferably at least 55%, more preferably at least 60%, even more preferably 70%, yet more preferably at least 75%, even yet more preferably 80%, even yet more than 85%, even yet more preferably at least 90%, even yet more preferably at least 95%, preferably at least 97%, most preferably at least 98% to the amino acid sequence of 3C-like proteinase [Severe acute respiratory syndrome coronavirus 2] having accession number YP_009725301, version number YP_009725301.1, 18 Jul. 2020 NCBI Protein Database GenPept [2021 Mar. 26 ]; available from https://www.ncbi.nlm.nih.gov/protein/1802476809.

Preferably, the viral encoded protease is a protease having at least 50% sequence similarity, preferably at least 55%, more preferably at least 60%, even more preferably 70%, yet more preferably at least 75%, even yet more preferably 80%, even yet more than 85%, even yet more preferably at least 90%, even yet more preferably at least 95%, preferably at least 97%, most preferably at least 98% to the amino acid sequence of 3C like protease (3 CL pro; R1A_SARS) at positions 3241-3546 having accession number P0C6U8 (Uniprot) Integrated into UniProtKB/Swiss-Prot: Sequence update: Jun. 10, 2008, Last modified: Feb. 10, 2021, Version 104 [2021 Mar. 26]; available from https://www.uniprot.org/uniprot/P0C6U8

Preferably, the viral encoded protease is a protease having at least 50% sequence similarity, preferably at least 55%, more preferably at least 60%, even more preferably 70%, yet more preferably at least 75%, even yet more preferably 80%, even yet more than 85%, even yet more preferably at least 90%, even yet more preferably at least 95%, preferably at least 97%, most preferably at least 98% to the amino acid sequence of 3C proteinase [Human rhinovirus A1] having accession number YP_009508983.1, version numberYP_009508983.1, 24 Aug. 2018 NCBI Protein Database GenPept [2021 Mar. 26]; https://www.ncbi.nlm.nih.gov/protein/YP_009508983.

Preferably, the viral encoded protease is a protease having at least 50% sequence similarity, preferably at least 55% more preferably at least 60%, even more preferably 70%, yet more preferably at least 75%, even yet more preferably 80%, even yet more than 85%, even yet more preferably at least 90%, even yet more preferably at least 95%, preferably at least 97%, most preferably at least 98% to the amino acid sequence of PEDV main proteinase [Porcine epidemic diarrhea virus] having accession number 4XFQ_A, PDB database, Deposited 28 Dec. 2014, Version 1.0 2016 Jan. 20 [2021 Mar. 26]; https://www.rcsb.org/structure/4XFQ.

The term “% sequence similarity” means that in a given alignment between two amino acid sequences, conservative substitutions of amino acid residues having similar physicochemical properties over a defined length of the alignment are allowed. Percentage similarity is readily determined by the skilled person, for example according to well known methods e.g. Curr Protoc Bioinformatics. 2013; 43: 3.5.1-3.5.9.

Preferably, the method has a specificity for viral infection over host inflammation response. Preferably the method has a selectivity for a specific virus or family of viruses.

Preferably, the viral infection is selected from the group consisting of Meningitis, gastro-intestinal infections, Summer flu, hand-foot-and-mouth, Poliovirus 3C Poliomyelitis, Common cold, asthma exacerbation in allergies, Encephalitis, Hepatitis A (chronic jaundice), Equine encephalitis, Smallpox, Rubella (German measles), Respiratory infection, Infant bronchiolitis, viral pneumonia, Acute respiratory syndrome, Pig disease, Yellow-fever, Hepatitis C, Pigs, cattle and sheep, disease, Acute upper respiratory, eye and intestinal tract infection, Herpes (systemic and topical) infection, Rabbit hemorrhagic Disease, Potato disease and Plant disease.

Preferably, the viral protease is a 3C protease and the infection is selected from the group consisting of Meningitis, gastro-intestinal infections, Summer flu, hand-foot-and-mouth, Poliovirus 3C Poliomyelitis, Common cold, asthma exacerbation in allergies, and Encephalitis.

Preferably, the viral protease is a 3C-like protease and the infection is selected from the group consisting of Infant bronchiolitis, viral pneumonia and Acute respiratory syndrome (SARS), COVID-19, Porcine epidemic diarrhea virus, feline infectious peritonitis, canine coronavirus infection, calf enteritis.

Preferably, the viral protease is serine protease and the infection is selected from the group consisting of small pox, equine encephalitis, respiratory infection, yellow fever. Hepatitis C, pigs, cattle and sheep disease.

The viral protease is preferably protease involved in proteolysis of a viral polypeptide.

Preferably, the method detects infection wherein the virus (causative agent) is selected from the group consisting of Picornaviridae, Coronaviridae, Adenoviridae, Coronaviridae and combinations thereof, most preferably the method detects infection wherein the virus (causative agent) is Coronaviridae. Even more preferably, the method detects infection wherein the virus (causative agent) is SARS, preferably SARS-CoV and/or SARS-CoV-2, even more preferably SARS-CoV-2 (2019 nCoV). The peptide defined herein has the advantage that it enables rapid detection of a viral encoded protease in a patient sample.

The embodiments described for the diagnostic peptide, apply mutatis mutandis to the aspect of the invention relating to the method/use of the peptide and kits/systems containing the same.

In another aspect of the invention, a method for the in vitro diagnosis of a viral infection in a subject is described, the method comprising the steps of:

i) contacting a sample of a bodily fluid/tissue sample with a peptide comprising a fluorescent agent having an emission wavelength of 650-900 nm and a non-fluorescent agent having an absorption wavelength of 650-900 nm, for quenching said emission of said fluorescent agent, and a cleavage site located between said fluorescent agent and first non-fluorescent agent, the cleavage site being specific for a viral protease,

ii) monitoring the fluorescence in the range of 650-900 nm from the peptide in step i),

wherein an increase in fluorescence in the range of 650-900 nm is indicative for the presence of a virus in the sample.

Preferably, the bodily fluid sample the bodily fluid sample is blood, saliva, sputum, broncheoalveolar fluid, tissue biopsy, and/or lacrimal fluid. Preferably, the bodily fluid sample is saliva.

The sample may be present on a swab or other collection device. Preferably, the bodily fluid/tissue sample is present on collection device. The collection device is preferably a swab.

In a preferred, the method comprises a step of contacting a collection device comprising a sample of bodily fluid/tissue with a buffer in a container.

Alternatively, the collection device is a container, preferably wherein the container is a vial or bottle for receiving saliva or sputum.

Optionally, the method comprises the step of centrifuging the sample to separate solid matter and/or precipitated biological material from a supernatant. Preferably, centrifugation can be at a force in the range of 1,000 g to 25,000 g, preferably, in the range of 3,000 g to 20,000 g, more preferably in the range of 3,700 g to 18,000 g. The centrifugation time is preferably in the range of 1 minute to 30 minutes, in the range of 2 to 20 minutes, more preferably in the range of 3 to 10 minutes. The supernatant or the aggregated matter resulting from centrifugation is preferably contacted with the reagent. In a preferred embodiment, the supernatant from centrifugation is preferably contacted with the reagent. In another preferred embodiment, the aggregated matter resulting from centrifugation is preferably contacted with the reagent.

Optionally, after addition of the reagent to the sample the mixture is agitated. The mixture is preferably agitated by aspiration with a pipette, vortexing or shaking.

In a particularly preferred embodiment, the sample is not subjected to a cell enrichment step prior to contacting the sample with the reagent. By “not subjected to a cell enrichment step” means there is not step of incubating the sample at 37° C. for a number of hours in order to increase the viral load of the sample. In other words, the sample can be transferred directly from the collection device to the container in which the reagent is added.

Preferably the sample is diluted in an assay buffer, preferably wherein the sample is diluted by a factor in the range of 1:10 to 1:10000, more preferably 1:100 to 1:1000.

Preferably, the assay buffer is capable of maintaining a pH in the range of 5-9, preferably about 6 to about 8, more preferably about 6.5 to about 7.5. preferably the buffer comprises HEPES, PIPES, Tris-Hydrochloride (Tris-HCl), or MOPS.

Preferably, the sample is contacted with a lysing agent. More preferably, the buffer contains a detergent that is capable of lysing the cellular material in the bodily fluid sample.

Preferably, the buffer comprises one or more non-ionic detergents, selected from the group consisting of N-octyl-D-glucopyranside, N-octyl-D-maltoside, ZWITTERGENT 3.14, deoxycholate; n-Dodecanoylsucrose; n-Dodecyl-D-glucopyranoside; n-Dodecyl-D-maltoside; n-Octyl-D-glucopyranoside; n-Octyl-p-D-maltopyranoside; n-Octyl-p-D-thioglucopyranoside; n-Decanoylsucrose; n-Decyl-p-D-maltopyranoside; n-Decyl-p-D-thiomaltoside; n-Heptyl-D-glucopyranoside; n-Heptyl-p-D-thioglucopyranoside; n-Hexyl-D-glucopyranoside; n-Nonyl-p-D-glucopyranoside; n-Octanoylsucrose; n-Octyl-D-glucopyranoside; n-Undecyl-D-maltoside; APO-10; APO-12; Big CHAP; Big CHAP, Deoxy; BRIJ® 35; d2E5; d2E6; Ci2E8; Ci2E9; Cyclohexyl-n-ethyl-p-D-maltoside; Cyclohexyl-n-hexyl-p-D-maltoside; Cyclohexyl-n-methyl-D-maltoside; Digitonin; ELUGENT™; GENAPOL® C-100; GENAPOL® X-080; GENAPOL® X-100; HECAMEG; MEGA-10; MEGA-8; MEGA-9; NOGA; NP-40; PLURONIC® F-127; TRITON® X-100; TRITON® X-I 14; TWEEN® 20; or TWEEN® 80 and mixtures thereof.

The buffer preferably comprises an ionic detergent selected from the group consisting of BATC, Cetyltrimethylammonium Bromide, Chenodeoxycholic Acid, Cholic Acid, Deoxycholic Acid, Glycocholic Acid, Glycodeoxycholic Acid, Glycolithocholic Acid, Lauroylsarcosine, Taurochenodeoxycholic Acid, Taurocholic Acid, Taurodehydrocholic Acid, Taurolithocholic Acid, Tauroursodeoxycholic Acid, TOPPA and mixtures thereof.

Preferably, the buffer comprises a zwitterionic detergent selected from the group consisting of amidosulfobetaines, CHAPS, CHAPSO, carboxybetaines, and methylbetaines.

Preferably, the buffer comprises an anionic detergent selected from group consisting of SDS, N-lauryl sarcosine, sodium deoxycholate, alkyl-aryl sulphonates, long chain (fatty) alcohol sulphates, olefine sulphates and sulphonates, alpha olefine sulphates and sulphonates, sulphated monoglycerides, sulphated ethers, sulphosuccinates, alkane sulphonates, phosphate esters, alkyl isethionates, sucrose esters and mixtures thereof.

The step of monitoring the increase in fluorescence in step iii) is preferably carried out in a detector adapted to receive a container comprising the sample and reagent. The detector preferably provides a readout in relative fluorescent units (RFU). The user can compare the readout to a control and a relative increase in RFU between the control and the sample is indicative of viral infection.

Preferably, the virus is a virus as described above. Preferably, the virus belongs to the family of Picornaviridae, or Coronaviridae.

The viral protease is preferably selected from the group consisting of Hepatitis encoded serine protease or metallo-protease (NS2 and NS3), Rhinovirus genome encoded cysteine proteases (3C and 2A), Coronavirus genome encoded cysteine protease (3CL), an Adenoviruses genome encoded serine-centered, neutral protease, a Retroviruses encoded aspartyl protease, preferably wherein the Coronavirus protease is a hSARS Coronavirus genome encoded cysteine protease, SARS coronavirus 19 main protease (EC number 3.4.22.69), more preferably the viral protease is a 3C-like (3CL) protease.

Preferably, a method for the in vitro diagnosis of a viral infection in a subject is described herein, the method comprising the steps of:

i) contacting a sample of saliva sample with a peptide comprising a fluorescent agent having an emission wavelength of 650-900 nm and a non-fluorescent agent having an absorption wavelength of 650-900 nm, for quenching said emission of said fluorescent agent, and a cleavage site located between said fluorescent agent and first non-fluorescent agent, the cleavage site being specific for a 3C-like protease,

ii) monitoring the fluorescence in the range of 650-900 nm from the peptide in step i),

wherein an increase in fluorescence in the range of 650-900 nm is indicative for the viral infection being selected from the group consisting of Infant bronchiolitis, viral pneumonia and Acute respiratory syndrome (SARS), COVID-19, Porcine epidemic diarrhea virus, feline infectious peritonitis, canine coronavirus infection, calf enteritis, preferably the viral infection is Acute respiratory syndrome (SARS), COVID-19, more preferably COVID-19.

In a further aspect, there is provided a kit for the diagnostic of a viral infection in a subject, comprising:

a) a container comprising the diagnostic peptide of formula (I),

b) a set of instructions for carrying out the diagnostic method defined herein.

In yet a further aspect, there is provided a system for the diagnosis of a viral infection in a subject, comprising:

a) a container for receiving a sample,

b) a container comprising the diagnostic peptide of formula (I),

c) a device adapted to receive the container and monitor the fluorescence signal emitted from the peptide when the peptide is contacted with the sample of bodily fluid.

The preferred embodiments for the in vitro method for diagnosing a viral infection described herein apply mutatis mutandis to the kit described herein.

In another aspect, there is provided a system for the diagnosis of a viral infection in a subject, comprising:

a) a container for receiving a sample,

b) a container comprising the diagnostic peptide of formula (I),

c) a device adapted to receive the container and monitor the fluorescence signal emitted from the peptide when the peptide is contacted with the sample of bodily fluid.

The preferred embodiments for the method for detecting a viral infection described herein apply mutatis mutandis to the system described herein.

Preferably, the device is a near-infrared spectrophotometer. Preferably the device is a point-of-care device.

The present invention has been described above with reference to a number of exemplary embodiments. Modifications and alternative implementations of some parts or elements are possible, and are included in the scope of protection as defined in the appended claims.

### EXAMPLES

### Example 1—Detection of SARS-CoV-19

A sample containing SARS-CoV-19 virus is contacted with a fluorescently quenched peptide (TVRLQSGF) comprising a fluorescent agent having an emission wavelength of 650-900 nm and a non-fluorescent agent having an absorption wavelength of 650-900 nm and a cleavage site specific for a Coronavirus (Ex 1.1). The negative control (scrambled) peptide is FQVLRS (Ex 1.2).

Measurements are taken using a hand held fluorimeter (DeNiro NIR Fluorimeter, DetactDiagnostics BV, The Netherlands), at 0, 1, 2 and 4 hours after contacting the reagent. The RFU for the control sample (buffer) was subtracted from each measurement.

An increase in RFU is detected or the sample but not for the negative control.

### Example 2

Synthetic peptides were purchased from CRB (Billingham, UK). The fluorescent agent was 10 Cy 7 (Cy7 DBCO from Click Chemistry Tools, Arizona, USA) and the non-fluorscent agent was QC-1 (QC-1 NHSester, LICOR, Nebraska, USA).

A stock solution of peptides in Millipure water was added to recombinant SARS-CoV-2 main protease (Mysourcebio, California, USA) to give a final concentration of peptide of 5 uM. The concentration of SARS-CoV-2 main protease varied from 0.2-2 uM. The buffer 30 used was 20 mM Tris, 100 mM NaCl, 1 mM EDTA, 1 mM DTT, pH 7.3 The fluorescence was monitored at 0, 5, 15, 30 and 45 minutes after addition of the peptide solution. The fluorescence was monitored using a DeNIRO NIR detector (DetactDiagnostics, Groningen, Netherlands).

As controls, subtilisin (Merck, Netherlands) and elastase (Sigma Aldrich, Netherlands) were used. The concentration and buffer for the control proteases was the same as for the experiments with SARS-CoV-2 main protease.

Relative activity was calculated according to the following calculation:

$$Activity units = 100 \times \frac{{RFU}_{2} - {RFU}_{1}}{{RFU}_{1}}$$

The results are shown in the Table 1 below:

TABLE 1

| | | Peptide | SARS-CoV-2 | |
|---|---|---|---|---|
| | | concen- | main | |
| | | tration/ | protease | Elastase |
| | Peptide | μM | 0.2 μM | 0.2 μM |
| 1 | Cy7-Ahx- | 5 | 13.7 | 7 |
| | [PEG₁]VLQS[PEG₁]K | | | |
| | (QC1)-CONH₂ | | | |
| 2 | Cy7-Ahx-[PEG₁]- | 5 | 13.1 | 0.1 |
| | HQS[PEG₁]K(QC1)- | | | |
| | CONH₂ | | | |
| 3 | Cy7-Ahx-AAFAA- | 5 | 0.4 | 2.2 |
| | K(QC1)-CONH₂ | | | |
| 1 | Cy7-Ahx- | 20 | 82.2 | 8.5 |
| | [PEG₁]VLQS[PEG₁]K | | | |
| | (QC1)-CONH₂ | | | |
| 2 | Cy7-Ahx-[PEG₁]- | 20 | 87.5 | 0.9 |
| | HQS[PEG₁]K(QC1)- | | | |
| | CONH₂ | | | |
| 3 | Cy7-Ahx-AAFAA- | 20 | 0.6 | 87.1 |
| | K(QC1)-CONH₂ | | | |

The ratio of activity units for SARS-CoV-2 main protease to elastase gives a measure of specificity for compound for a particular protease. The ratio of activity units is shown below:

TABLE 2

| | | Ratio for |
|---|---|---|
| | | SARS- |
| | | CoV-2 main |
| | | protease to |
| | Peptide | elastase |
| 1 | Cy7-Ahx-[PEG₁]VLQS[PEG₁]K(QC1)- | 3.5 |
| | CONH₂ | |
| 2 | Cy7-Ahx-[PEG₁]-HQS[PEG₁]K(QC1)- | 54 |
| | CONH₂ | |
| 3 | Cy7-Ahx-AAFAA-K(QC1)-CONH₂ | 0.15 |

### Example 3—Mass Spectrometric Characterization

Analysis of peptides 1-5 was carried out using RP-HPLC-MS. Mass spectra were acquired on a Agilent Technologies 6530 Accurate Mass Q-TOF LC/MS with an Agilent Infinity 1260/1290 HPLC. RP-HPLC was carried out using a RP-aqueous 030 analytical column, 5 uM, 150×2.0 mm [Phenomenex]. The mobile phase was A) 10 mM ammonium formate/2% v/v MeCN, pH 6.2 and B) 10 mM ammonium formate/90% v/v MeCN, pH 6.2.

The gradient used was 0.2 mL-min, 25 minutes.

0.1 mg/mL solution of each peptide in Table 3 was incubated with SARS-CoV-2 main protease (at 10 μM).

TABLE 3

| | Starting material | | | |
|---|---|---|---|---|
| | (measured mass) | | Cleavage fragment | |
| Peptide | 0 min | 4 hours | (measured mass) | |
| 1 | 1077.05 | Not | C₆₃H₈₉CIN₈O₁₈S₄ | 350.97 |
| | [M − 3H]⁻³ | measured | | |
| 2 | 1052.35 | Not | C₈₅H₁₀₁N₁₂O₂₂S₄ | 297.17 |
| | [M − 3H]⁻³ | measured | | [M − 6H]⁻⁶ |
| 3 | 1062.70 | 1062.70 | NONE | NONE |
| | [M − 3H]⁻³ | [M − 3H]⁻³ | | |

Cleavage of peptide 1 took place between Q-S. Cleavage of peptide 2 took place between Q-S. No cleavage was observed for peptide 3.

0.1 mg/mL solution of each peptide in Table 4 was incubated with elastase (at 2 μM). The results are shown in Table 4.

TABLE 4

| | Starting material | | | |
|---|---|---|---|---|
| | (measured mass) | | Cleavage fragment | |
| Peptide | 0 min | 4 hours | (measured mass) | |
| 1 | 1077.05 | 1077.05 | — | — |
| | [M − 3H]⁻³ | [M − 3H]⁻³ | | |
| 2 | 1052.35 | 1077.05 | — | — |
| | [M − 3H]⁻³ | [M − 3H]⁻³ | | |
| 3 | 1062.70 | 1062.70 | C₂₁H₂₃N₄O₅ | 419.23 |
| | [M − 3H]⁻³ | [M − 3H]⁻³ | | [M − H]−1 |

### Example 4—Clinical Data

Clinical samples that had been characterized as either positive or negative for SARS-CoV-2 by PCR (Corman et al. Detection of 2019 novel coronavirus (2019-nCoV) by real-time RT-PCR. Euro Surveill. 2020; 25(3):pii=2000045) were used (StreekLab, Haarlem, The Netherlands). Buffer A was PBS, pH 7.2

Stock solution of the substrate was prepared by adding 1 mL of buffer A to a final concentration of 0.1 mg/mL=26 uM).

Blank (peptide solution only)

50 μL of peptide stock was added to 200 μL of buffer A and vortexed.

The ‘Blank’ was measured on the DeNIRO.

Sample Measurement

100 μL buffer A was added to an 500 μl Eppendorf, The sample bottle was vortexed, 100 μL of sample taken and added to the Eppendorf. 50 μL of peptide stock (final concentration 20 uM) was added. The Eppendorf was vortexed 2×5 seconds and immediately the fluorescence was measured on the DeNIRO. The Eppendorf was then left at room temperature and remeasured at 5, 10, 15, 30, 60 minutes. Results are shown in Table 5. A relative activity of >10% indicates the presence of SARS-CoV-2 (+), a relative activity of <10% indicates absence of SARS-CoV-2 (−).

TABLE 5

| | | Peptide 2 |
|---|---|---|
| Sample | Peptide 1 | (20 μM) |
| SARS-CoV-2 positive (swab) | + | + |
| SARS-CoV-2 positive (saliva) | + | + |
| SARS-CoV-2 negative (swab) | − | − |
| SARS-CoV-2 negative (saliva) | − | − |
| Rhinovirus positive | − | − |
| Mycobacterium tuberculosis positive | − | − |

### Example 5—SARS-CoV-2 Test

Peptide 1 and Peptide 2, as described in Example 2, as well as Peptide 4 (Cy7-Ahx-[PEG1]-FHT-[PEG1]K[(QC1)-CONH₂) were tested in nasopharyngeal (NP) samples of subjects suspected to be infected with SARS-CoV-2. Samples were previously used to run PCR tests to detect presence of infection. Samples were NP swabs in M4 viral transport media. All samples were stored for max. 48 hours at 4 degC prior to analysis.

All samples were subjected to confirmatory PCR using the method according to Corman et al. Detection of 2019 novel coronavirus (2019-nCoV) by real-time RT-PCR. Euro Surveill. 2020; 25(3):pii=2000045. https://doi.org/10.2807/1560-7917.ES.2020.25.3.2000045. CT values for the positive samples ranged from 16 to 33.8. One sample with a CT>35 was not included.

Peptide stock solutions were prepared by dissolving peptide 1, 2 and 4 respectively in buffer A (PBS, pH 7.2) to a final concentration of 61.86 μM.

Samples were monitored using a DeNIRO near-infrared fluorometer (DetactDiagnostics 5 BV, The Netherlands).

Negative control: peptide solution only 1.81 μL of peptide stock (1, 2 or 4) was added to 169 μL buffer A, vortexed, and measured ‘Blank’ in DeNiro.

Sample measurement [total volume in Eppendorf sample+buffer+substrate=250 μL)
1. 69 μL buffer A was added to an 0.5 mL Eppendorf
2. Copan swab bottle was briefly vortexed, 100 μL sample liquid removed and added to the Eppendorf
3. 81 μL of CoviTact peptide stock was added to the Eppendorf (final concentration 20 μM peptide)
4. Vortexed 2×5 seconds
5. Measured straight away [0 minutes] and at 5 minutes.

Results are presented in Table 6.

TABLE 6

| no. samples | Peptide 1 | Peptide 2 | Peptide 4 |
|---|---|---|---|
| True positive (TP) | 11 | 30 | 7 |
| False negative (FN) | 19 | 3 | 23 |
| Total | 30 | 33 | 30 |
| False positive (FP) | 9 | 6 | 4 |
| True negative (TN) | 21 | 30 | 26 |
| Total | 30 | 36 | 30 |
| Total TP + FP | 20 | 36 | 11 |
| Total FN + TN | 40 | 33 | 49 |

Table 7 shows the positive and negative predictive values, which were calculated as follows:

Positive predictive value=100*TP/(TP+FP)

Negative predictive value=100*TN/(TN+FN)

Overall percent agreement=100*(TP+TN)/total

TABLE 7

| | Positive | Negative | Overall |
|---|---|---|---|
| | predictive | predictive | percentage |
| | value | value | agreement |
| Peptide 1 | 55% | 52.5% | 53.3% |
| Peptide 2 | 83.3% | 90.9% | 87.0% |
| Peptide 4 | 63.6% | 53.1% | 55.0% |

Peptide 2, according to the invention, shows a high (about 90%) correlation with confirmed presence/absence of infection.

The peptide content of peptides according to the present invention and those of the prior art are compared in table 8.

TABLE 8

| | Relative | % | Cross |
|---|---|---|---|
| | molecular | peptide | reactivity |
| | mass | ([b]) of | to saliva |
| Peptide | (RMM) | total | proteases |
| 1 | 3234.15 | 13 | − |
| 2 | 3160.05 | 11 | − |
| US201014388, | 1582.33 | 74.6 | + |
| Example 2 | | | |
| SEQUENCE 113 | | | |
| US 2007/160981 | 1605 | 77 | + |
| (Ex. 3) | | | |
| WO2013/049382 | 2214 | 71 | + |
| (Table 5; SARS CoV 2) | | | |
| WO2005/017191 | 1594 | 74 | + |
| (Rhinovirus, SEQ ID 113) | | | |

− Not observed;
+ observed

1.-18. (canceled)

19. A method for in vitro diagnosis of a viral infection in a subject, the method comprising the steps of:
(i) contacting a sample of a bodily fluid with a peptide comprising a fluorescent agent having an emission wavelength of 650-900 nm and a non-fluorescent agent having an absorption wavelength of 650-900 nm, for quenching said emission of said fluorescent agent, and a cleavage site located between said fluorescent agent and first non-fluorescent agent, the cleavage site being specific for a viral protease;
(ii) monitoring the fluorescence in the range of 650-900 nm from the peptide in step (i), wherein an increase in fluorescence in the range of 650-900 nm is indicative for the presence of a viral protease in the sample.

20. The method according to claim 19, wherein the bodily fluid sample is blood, saliva, sputum, broncheoalveolar fluid, tissue biopsy, and/or lacrimal fluid.

21. The method according to claim 19, wherein the viral protease is selected from the group consisting of Hepatitis encoded serine protease or metallo-protease (NS2 and NS3), Rhinovirus genome encoded cysteine proteases (3C and 2A), Coronavirus genome encoded cysteine protease (3CL), an Adenoviruses genome encoded serine-centered, neutral protease, a Retroviruses encoded aspartyl protease, preferably wherein the Coronavirus protease is a SARS Coronavirus genome encoded cysteine protease, SARS coronavirus 19 main protease (EC number 3.4.22.69).

22. The method according to claim 19, wherein the peptide is represented by formula (I)
[a]-[b]-[c] (I)
wherein:
[a] is a fluorescent agent having an emission wavelength of 650-900 nm,
[b] is a peptide comprising the amino acid sequence Xₐₐ₁, Xₐₐ₂, Xₐₐ₃
wherein Xₐₐ₁ is a hydrophobic or basic amino acid,
wherein Xₐₐ₂ is a polar, neutral or basic amino acid,
wherein Xₐₐ₃ is a polar, neutral or basic amino acid,
[c] is a non-fluorescent agent having an absorption wavelength of 650-900 nm, for quenching said emission of said fluorescent agent,
wherein Xₐₐ₁, Xₐₐ₂, Xₐₐ₃ represents a cleavage site for a viral encoded protease, wherein cleavage of the cleavage site results in release of the non-fluorescent agent from the peptide, wherein release of the non-fluorescent agent is indicative for the presence of a virus, preferably wherein [b] represents at most 50% by weight of the total mass of the diagnostic peptide, based on the total mass of [a]-[b]-[c].

23. The method according to claim 22, wherein the peptide represented by formula (Ia)
[a]-[linker₁]-[b]-[-linker₂]-[c] (Ia)
wherein the linkers are independently selected from the group consisting of an optionally substituted hydrocarbyl group, and a non-proteolytic hydrocarbyl group.

24. The method according to claim 23, wherein the hydrocarbyl linker is selected from the group consisting of beta-alaninyl, 4-aminobutyrl, 2-(aminoethoxy)acetyl, 3-(2-aminoethoxy)propanyl, 5-aminovaleryl, 6-aminohexyl, 8-amino-3,6-dioxaoctanyl and 12-amino-4,7,10-trioxadodecanyl, preferably 6-aminohexyl.

25. The method according to claim 19, wherein Xₐₐ₁ is selected from the group consisting of alanine, leucine, valine, norleucine, norvaline, isoleucine, isovaline, alloisoleucine, phenylalanine, histidine, arginine and lysine, preferably from the group consisting of histidine, arginine and lysine, preferably histidine.

26. The method according to claim 19, wherein Xₐₐ₂ is selected from the group consisting of histidine, asparagine and glutamine, preferably glutamine.

27. The method according to claim 19, wherein Xₐₐ₃ is selected from the group consisting of serine, threonine and glycine, preferably, wherein Xₐₐ₃ is serine.

28. The method according to claim 19, wherein the peptide comprises a sequence selected from HQS, RQS or KQS.

29. The method according to claim 19, wherein the infection is selected from the group consisting of infant bronchiolitis, viral pneumonia, Acute respiratory syndrome (SARS), COVID-19, Porcine epidemic diarrhea virus, feline infectious peritonitis, canine coronavirus infection and calf enteritis.

30. The method according to claim 19, wherein the sample is diluted in an assay buffer, preferably wherein the sample is diluted by a factor in the range of 1:10 to 1:10000, more preferably 1:100 to 1:1000.

31. The method according to claim 19, wherein the sample is contacted with a lysing agent.

32. A system for the in vitro diagnosis of a viral infection in a subject, comprising:
a) a container for receiving a sample of bodily fluid,
b) a container comprising a peptide is represented by formula (I)
[a]-[b]-[c] (I)
wherein:
[a] is a fluorescent agent having an emission wavelength of 650-900 nm,
[b] is a peptide comprising the amino acid sequence Xₐₐ₁, Xₐₐ₂, Xₐₐ₃
wherein Xₐₐ₁ is a hydrophobic or basic amino acid,
wherein Xₐₐ2 is a polar, neutral or basic amino acid,
wherein Xₐₐ₃ is a polar, neutral or basic amino acid,
[c] is a non-fluorescent agent having an absorption wavelength of 650-900 nm, for quenching said emission of said fluorescent agent,
wherein Xₐₐ₁, Xₐₐ₂, Xₐₐ₃ represents a cleavage site for a viral encoded protease, wherein cleavage of the cleavage site results in release of the non-fluorescent agent from the peptide, wherein release of the non-fluorescent agent is indicative for the presence of a virus, preferably wherein [b] represents at most 50% by weight of the total mass of the diagnostic peptide, based on the total mass of [a]-[b]-[c], and
c) a device adapted to receive the container and monitor the fluorescence signal emitted from the peptide when the peptide is contacted with the sample of bodily fluid.

33. A diagnostic peptide represented by formula (Ib)
[a]-[linker₁]-[b]-[-linker₂]-[c] (Ib)
wherein:
[a] is a fluorescent agent having an emission wavelength of 650-900 nm,
[b] is a peptide comprising 3-10 amino acids and having the amino acid sequence Xₐₐ₁, Xₐₐ₂, Xₐₐ₃
wherein Xₐₐ₁ is histidine, arginine or lysine,
wherein Xₐₐ₂ is glutamine,
wherein Xₐₐ₃ is a polar, neutral or basic amino acid,
[c] is a non-fluorescent agent having an absorption wavelength of 650-900 nm, for quenching said emission of said fluorescent agent,
[linker₁] and [linker₂] are independently selected from the group of an optionally substituted hydrocarbyl group and a non-proteolytic hydrocarbyl group
wherein Xₐₐ₁, Xₐₐ₂, Xₐₐ₃ represents a cleavage site for a viral encoded protease, wherein cleavage of the cleavage site results in release of the non-fluorescent agent from the peptide, wherein release of the non-fluorescent agent is indicative for the presence of a virus belonging to the family of Coronaviridae, preferably wherein the virus is SARS-Cov-2 (SARS-COV-19).
